(19) United States
(12) Patent Application Publication
(10) Pub. No.: US 2014/0155743 A1
(43) Pub. Date: Jun. 5, 2014

(54) IMAGING USING SETS OF CARBON NANOTUBES

(71) Applicants: Balasundar Iyyavu Raju, Chester, NY (US); Lucian Remus Albu, Forest Hills, NY (US)

(72) Inventors: Balasundar Iyyavu Raju, Chester, NY (US); Lucian Remus Albu, Forest Hills, NY (US)

(73) Assignee: KONINKLIJKE PHILIPS N.V., EINDHOVEN (NL)

(21) Appl. No.: 14/234,446

(22) PCT Filed: Jul. 23, 2012
(86) PCT No.: PCT/IB2012/053736

§ 371 Date: Jan. 23, 2014

Related U.S. Application Data
(60) Provisional application No. 61/521,432, filed on Aug. 9, 2011.

(51) Int. Cl. A61K 49/22; A61B 5/00

(52) U.S. Cl. CPC A61K 49/222; A61B 5/0095
USPC 600/431

(57) ABSTRACT

An imaging method uses a plurality of sets of carbon nanotubes. Within a set the carbon nanotubes carry markers for a respective receptor that is specific for the set and the carbon nanotubes have a geometry, characterized for example by a chiral number that gives rise to an electromagnetic absorption peak at a wavelength specific to the set. An image is formed by transmitting electromagnetic radiation to a body, substantially at the wavelengths of the absorption peaks of the sets, e.g. time multiplexed with each other, and detecting for example an ultrasound response to absorption of the transmitted electromagnetic radiation. Different images of the electromagnetic absorption as a function of position in the body are formed for different wavelengths.

### FIELD OF THE INVENTION

The invention relates to an imaging method and system using sets of carbon nanotubes, a contrast agent composition for imaging, and a kit of parts for forming a contrast agent.

### BACKGROUND

U.S. Pat. No. 7,500,953 describes contrast agents for photo-acoustic imaging. Photo-acoustic imaging involves the excitation of acoustic (ultrasound) waves in a body of material by means of irradiation with light pulses. The light pulses lead to position dependent heating, as a function of local light absorption properties. In turn this results in the excitation of sound waves with excitation amplitudes that depend on position. After the waves have travelled through the body, the sound amplitude is detected as a function of time and position, and from this an image of absorption as a function of position is reconstructed.

U.S. Pat. No. 7,500,953 describes the use of nanoparticles as a contrast agent for absorbing energy from the light pulses. Nanoparticles are selected on the basis of size or shape so that they are tuned to the same wavelength of irradiating light. The selected nanoparticles are applied to a patient for example, after which an image of the distribution of the nanoparticles through the patient's body can be obtained by means of photo-acoustic imaging using pulses of light at the wavelength of the nanoparticles. U.S. Pat. No. 7,500,953 describes a wide range of possible nano-particles, including any metal, metal alloy, or combinations of metals and non-metals. Gold, silver, palladium, and platinum and carbon nano tubes are mentioned, and the particles may be filled with water, nitrogen, argon, or neon, aqueous gels, and organic substances.

U.S. Pat. No. 7,500,953 describes various further embodiments of this technique. One such embodiment involves attaching the nanoparticles to markers for specific receptor molecules in the body. In this way the nanoparticles will be concentrated in body regions where the receptor molecules are present. Hence the resulting image will show the concentration of the receptor molecules as a function of position.

In a different embodiment, U.S. Pat. No. 7,500,953 describes the use of contrast agents that are sensitive to two different irradiation wavelengths, by using a mixture of nanoparticles with different shape, composition, and dimensions. The shape, composition, and dimensions of a first set of nanoparticles is chosen corresponding to a wavelength that is especially useful for detecting vascular tumors rich in hypoxic blood. The shape, composition, and dimensions of a second set of nanoparticles is chosen corresponding to a wavelength that is highly penetrating and is especially sensitive to tumors and other tissues containing deoxygenated blood. Comparison of the images obtained with these different wavelengths provides for differentiation between diseased tissue and either normal tissue or abnormal, but harmless, tissue.

### SUMMARY OF THE INVENTION

Among others, it is an object to provide for an imaging method and system and a contrast agent that make it possible to obtain more detailed image information.

An imaging method according to claim 1 is provided. This method uses a plurality of sets of carbon nanotubes, respective ones of the sets each comprising carbon nanotubes carrying markers for a respective receptor in a body of material, different from markers of the carbon nanotubes in other ones of the sets, or in a different combination of concentrations of markers as in the other ones of the sets, the carbon nanotubes of the respective one of the sets having a respective geometry, for example a respective chiral number, giving rise to an absorption peak for electromagnetic radiation at a wavelength different from the wavelengths of the absorption peaks corresponding to the geometries of the carbon nanotubes in the other ones of the sets. The method may be applied for example after administration of the combination of carbon nano-tubes from each of these sets to the body, as one composition of carbon nanotubes, and/or by realizing the administration of the combination by administering carbon nanotubes from individual sets separately so that they may be present in the body simultaneously. Also use may be made of carbon nanotubes that are already present in the body. The use of carbon nanotubes provides for a wide range of different geometries, e.g. different chiral numbers, that provides for a considerable number (at least three or more) of different carbon nanotubes that can selectively be made to absorb electromagnetic radiation by using different wavelengths.

Electromagnetic radiation is transmitted to the body, substantially at the wavelengths of the absorption peaks of the sets, multiplexed with each other. The transmitted electromagnetic radiation may be infrared radiation with wavelengths in a range of 0.7-1.1 micrometer for example, but other wavelengths could be used. The wavelengths may be multiplexed by time division multiplexing for example, electromagnetic radiation of different wavelengths being transmitted at different time points, but other multiplexing techniques such as modulation frequency multiplexing may be used.

A response to absorption of the transmitted electromagnetic radiation is detected for example in the form of ultrasound. Other responses such as inelastically scattered radiation could be used instead (e.g. Raman spectroscopy).

From the detected response to the radiation one or more images of the absorption as a function of position in the body may be formed. When the electromagnetic radiation is visible light and ultrasound amplitude as a function of time is detected, the one or more images will be photo-acoustic images, but similar image forming techniques may be used with electromagnetic radiation at other wavelengths.

A plurality of images may be formed for respective ones of the plurality of the wavelengths, each from the response to absorption of electromagnetic radiation at a respective one of the wavelengths. When the carbon nanotubes of different sets carry mutually different markers, each image shows the effect of binding of a different marker. These images may be combined to form a combination image for selected ones of the sets. Also combination images for selected combinations of the wavelengths may be formed directly, without images from individual wavelengths, by combining detections for the selected combinations of the wavelengths and forming the images from the combined detections. Even if the set carbon nanotubes contains carbon nanotubes with mutually different markers that are the same as markers of carbon nanotubes in different sets, a difference in the combination of concentrations of the carbon nanotubes that carry different markers, compared to the combination of concentrations in other sets still may provide for images with different information.

In an embodiment a pulse is transmitted at the wavelength of at least one of the sets, with a higher energy than the electromagnetic radiation used for imaging. The higher energy pulse may be used to detach the markers from the carbon nanotubes. This can be used as a “reset”, prior to addition of other nanotubes, or as a trigger for measuring time dependent responses.

In an embodiment further sets of carbon nanotubes are used, similar to the set of carbon nanotubes with markers, but with releasable substances such as drugs instead of or in addition to the markers. The carbon nanotubes in the further sets may have the same associated absorption wavelengths as those in the earlier mentioned sets or different absorption wavelengths. They may be used to trigger release of selected substances by means of transmission of electromagnetic radiation to the body, substantially at the wavelengths of the absorption peaks of a selected one or ones of the further sets. Thus for example a treatment selected based on the images may be applied immediately after imaging.

### BRIEF DESCRIPTION OF THE DRAWING

These and other objects and advantageous aspects will become apparent from a description of exemplary embodiments, with reference to the following FIGURE.

FIG. 1 shows an imaging system

### DETAILED DESCRIPTION OF EXEMPLARY EMBODIMENTS

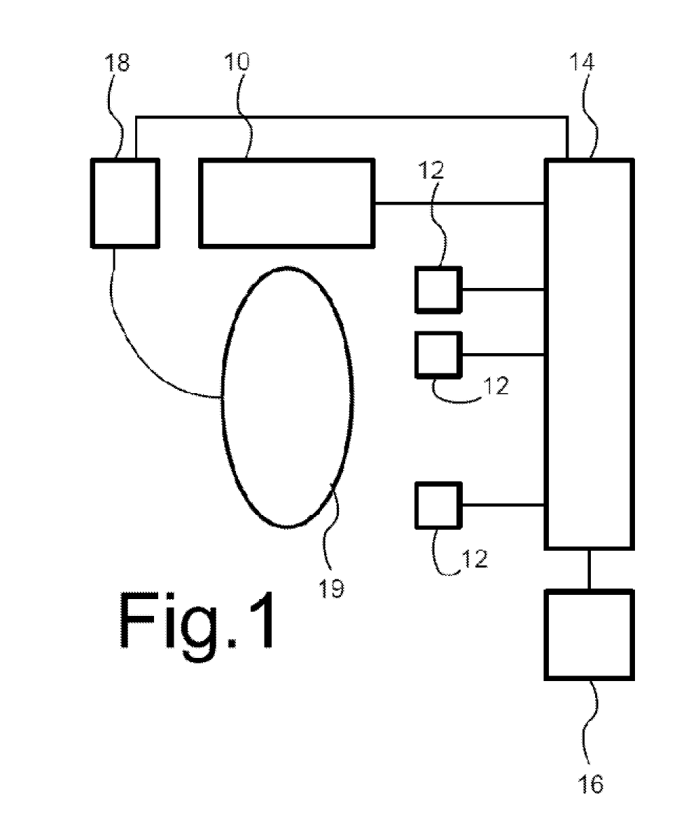

FIG. 1 shows an imaging system comprising an electromagnetic radiation source 10, an array of ultrasound receivers 12, a processing system 14 and an image display device 16. Processing system 14 is coupled electromagnetic radiation source 10, ultrasound receivers 12 and image display device 16. The imaging system may also comprise a contrast agent supply device 18, optionally with a control input coupled to processing system 14.

In operation electromagnetic radiation source 10 and array of ultrasound receivers 12 are directed at a body of material 19 containing contrast agent. The contrast agent may be supplied to the body 19 from contrast agent supply device 18 for example. Processing system 14 causes electromagnetic radiation source 10 to emit pulses of electromagnetic radiation to body 19 at a plurality of successively different wavelengths. This may be done for example after supply of contrast agent has been stopped. Infrared radiation at wavelengths in a range of 0.7-1.1 micrometer may be used for example. Ultrasound receivers 12 receive ultrasound from body 19, and supply resulting detection signals to processing system 14.

Processing system 14 uses the detected signals to compute electromagnetic-acoustic (e.g. photo-acoustic) images of body 19, each for a respective one of the wavelengths, e.g. for a respective one of the pulses. Preferably, at least three different wavelengths are used to compute at least three images respectively. Methods of computing photo-acoustic images are known per se. For each wavelength, the pulse leads to reception of ultrasound with a time-dependent amplitude. The amplitudes received at respective time delays with respect to the pulse correspond to pulse absorption in a respective virtual surface relative to the ultrasound receiver 12 that receives the amplitude. Ultrasound receivers 12 at different positions define different (intersecting) virtual surfaces. The full position dependence of the absorption can be reconstructed by combining the amplitudes received by ultrasound receivers 12 at different positions, for example by means of back projection.

Optionally, a plurality of pulses at the same wavelength may be used to form an image. The results obtained with different pulses may be averaged for example, to improve the signal to noise ratio. Also, the position of ultrasound receivers 12 may be changed between different pulses so as to detect the ultrasound response at more positions. Furthermore, a scan may be performed, wherein electromagnetic radiation source 10 concentrate energy of the pulses successively in different virtual surfaces of body 19 to help resolve the position dependence of the absorption. When a plurality of pulses at the same wavelength may be used to form an image, these pulses may be interleaved with pulses at other wavelengths that are used to form other images. Thus for example, when position of ultrasound receivers 12 is changed to detect the ultrasound response at more positions, pulses at one wavelength may be interleaved with pulses at other wavelengths. Thus, no additional movement is needed to perform measurements with the pulses at the other wavelengths. Similarly, successive pulses at a plurality of wavelengths may be used in each step in a scan of radiation source 10.

In an embodiment, processing system 14 may be configured to cause the detected images for individual wavelengths (e.g. at least three different wavelengths) to be displayed. The images may be stored in a storage device (not shown). In another embodiment processing system 14 combines the detected images for respective different wavelengths into a combination image and causes image display device 16 to display the combination image. Combined images that combine at least three different wavelengths may be realized by adding images for individual wavelengths, thresholding images for individual wavelengths (determining a yes/no value whether a pixel value is above a threshold value) and performing logic operations on the result, segmenting the images for individual wavelengths into segments wherein pixels with similar values are always at no more than a maximum distance from each other and combining such segments (taking cross-sections, junctions etc).

The contrast agent comprises carbon nanotubes with attached markers that bind selectively to specific reflectors in body 19. As is known per se, a carbon nanotube can be thought to arise from folding of a graphene sheet, which is a planar lattice structure of hexagonal carbon rings, resembling chicken wire. The chiral vector Cₕ of such a nano-tube is represented by a combination (n, m) of integer numbers n, m, of the respective unit vectors of the lattice along one revolution of the nanotube. It has been found that carbon nano tubes have sharp electromagnetic absorption peaks for electromagnetic radiation at frequencies (wavelengths) that differ dependent on the numbers n, m. Single and/or double walled carbon nanotubes could be used.

The contrast agent comprises a mix of sets of carbon nanotubes. The carbon nanotubes in each set all have the chiral vector (n,m) associated with that set. The chiral vectors associated with different sets are different from one another. The carbon nanotubes of different sets have absorption peaks at mutually different frequencies. The use of carbon nanotubes with different chiral vectors makes is possible to realize a considerable number of sets (at least three) with mutually different associated wavelengths, but with similar size, shape and dimension. Absorption wavelength bandwidths of 100 nm can be realized.

Furthermore, each set is associated with a respective marker molecule that binds selectively to corresponding receptors in body 19. Nanotubes in the set carry the marker molecule that is associated with set. Different sets are associated with different markers. Optionally, at least one of the sets is not associated with any marker, nanotubes in that set carrying no marker molecule.

The markers may include
An antibody, including monoclonal antibodies, for example antibodies targeted to CD20 epitopes expressed on tumor cells and/or antibodies to target growth factors such as VEGF, FGF, HGF present in the neovasculature of tumor environment
A peptide, for example RGD (arginine-glycine-aspartic acid) peptide
A vitamin, for example folic acid binding to folate receptors expressed by many tumors
An aptamer, for examples a strand of oligonucleotides

Attachment of such markers to carbon nanotubes (functionalization) per se is described in an article titled “Carbon nanotubes as multifunctional biological transporters and near-infrared agents for selective cancer cell destruction”, by Nadine Wong Shi Kam et al, publishes in PNAS Vol 102 no 33, pages 11600-11605 and in an article titled “Cell-penetrating CNTs for delivery of therapeutics”, by Lara Lacerda, Simona Raffab, Maurizio Pratoc, Alberto Biancod, Kostas Kostarelosa, published in Nanotoday, December 2007, Volume 2, NUMBER 6A pages 38-43. Kam (2005) discloses functionalization of SWNT (single wall carbon nanotube) with a folate moiety, selective internalization of SWNTs inside cells labeled with folate receptor tumor markers. Kang (2008) discloses the uptake of folate conjugated nanotubes inside Hep G2 cells that overexpress folate receptor on the surface of cell. Recently, McDevitt et al have reported a successful multiple derivatization of CNTs with a monoclonal antibody used as a targeting ligand. The team constructed a CNT-antibody conjugate specifically to target the CD20 epitope on Human Burkitt lymphoma cells

As in U.S. Pat. No. 7,500,953 markers could be amino acids, peptides, oligopeptides, polypeptides, proteins, antibodies, antibody fragments, hormones, hormone analogues, glycoproteins, lectins, sugars, saccharides, including monosaccharides and polysaccharides, carbohydrates, vitamins, steroids, steroid analogs, hormones, cofactors, and genetic material, including nucleosides, nucleotides, nucleotide acid constructs and polynucleotides and derivatives of these materials.

Attachment of markers to carbon nano-tubes is also disclosed per se in US 2008/227687). Basically, a set of carbon nanotubes may be formed by mixing carbon nanotubes with the same geometry and chiral vector with a solution containing a specific type of marker molecules. Subsequently, the nanotubes of different sets with attached markers may be mixed and supplied to body 19, or supplied to body 19 so that they mix in body 19.

The sets of nanotubes may be administered orally to a human or to an animal for example, by swallowing and/or inhalation, and/or by subcutaneous and/or intravenous injection, and/or from a device for administration at a slower rate (drip).

The wavelengths of different ones of the pulses from electromagnetic radiation source 10 each substantially equal the wavelength of the absorption peaks of the carbon nanotubes of a respective different one of the sets (i.e. if they are not exactly at the peak, at their distance to the peak position the absorption is no less than one quarter of the absorption due to the set of carbon nanotubes at the peak and preferably no less than one half). At least three different wavelengths that are substantially at the peaks of three different types of carbon nanotubes are preferably used.

Electromagnetic radiation source 10 may comprise a set of lasers tuned to different ones of these wavelengths for example, or a broadband pulse source combined with one or more monochromators that are tuned to the wavelengths of the absorption peaks of the carbon nanotubes of the respective different ones of the sets. Absorption wavelengths of carbon nano tubes with different chiral numbers are known per se, for example from Kataura plots. The absorption wavelengths may also be weakly dependent on the environment of the carbon nanotubes. In an embodiment the received is optimized signal by initially irradiating body 19 with test signals at a plurality of slightly different wavelengths (including one that is based on calculations or a priori empirical data), selecting one of these plurality of wavelengths where the received signal is strongest and using the selected wavelength for the excitation of the ultrasound waves from which the image is formed.

Because images are obtained by excitation with electromagnetic pulses at different wavelengths, each of which absorbed by a respective set of carbon nano-tubes, different images show concentration of carbon nano-tubes with a different marker.

In one embodiment the contrast agent with a mix of different sets is applied first and the application is stopped. After stopping a wait period is observed that is sufficiently long for the contrast agent to reach a target area in body 19 and leave the target area when the markers are not bound to receptors in the target area. The pulses are applied after this wait period. Hence the resulting images will show concentration of carbon nano-tubes with markers bound to receptors.

In another embodiment the contrast agent with a mix of different sets may be applied continuously during application of the pulses. Hence the resulting images will show a carbon nano-tubes at each wavelength, but with an increase of the concentration of a selected set or sets of carbon nano-tubes in areas where these carbon nano-tubes are bound to receptors. In this embodiment difference images may be used, for example each the difference between an image formed using a pulse at the wavelength of a respective set of carbon nano-tubes with a respective marker and an image formed with a pulse at the wavelength of a set of carbon nano-tubes without marker. Alternatively the difference may be taken between images formed using pulses at the wavelengths of different sets of carbon nano-tubes, each with a different marker.

The mix of contrast agents may be realized first mixing a selection of separate solutions that each contains a marker carried only by carbon nanotubes that absorb electromagnetic radiation in an absorption peak distinct from absorption peaks of carbon nanotubes carrying markers in the other solutions. The result of this mixing may be fed to body 19. Alternatively, the mix may be applied by applying the solutions separately to body 19 with a relative timing such that the carbon nanotubes from different solutions will be present simultaneously in body 19. The set of solutions forms a kit of parts from which a mix can be composed.

The images may be used in different ways. For example, they may be used to distinguish image areas where the sets of carbon nanotubes are bound to receptors of a plurality of predetermined receptor types. As another example, the images may be used to distinguish image areas where the sets of carbon nanotubes are bound to receptors of a first predetermined receptor type (or optionally a first plurality of predetermined receptor types) but not to receptors of a second predetermined receptor type (or optionally a second plurality of predetermined receptor types).

Although embodiments have been described wherein images are formed, if not displayed, for all the sets of carbon nanotubes, it should be appreciated that instead detection results for different wavelengths may be combined before the images are formed, so that only a combination image is formed. The detected ultrasound amplitudes for corresponding delays relative to pulse of electromagnetic irradiation at different wavelengths may be added and/or subtracted for example and the image may be formed from the added and/or subtracted image. Thus for example an image for a sum of selected sets may be computed.

The images obtained for individual sets may be displayed, for example dependent on a selection received by processing system 14 from an operator. Alternatively an image may be generated and displayed based on a combination of images obtained for different sets, for example using the images obtained for individual sets in different color channels of the combined image, or using a difference between images obtained for different sets in the combined image, or showing image parts of an image combined with a first set only where image values obtained for a second set are above a threshold, or below a threshold etc.

If the amplitude of a pulse is raised and/or its duration is extended, this may result in absorption of so much energy that the markers will become detached from the carbon nano-tubes. Typically, the energy is proportional to a product of the duration and the square of the amplitude. The energy, or the combination of amplitude and duration, at which this happens can easily be determined by transmitting test pulses with different combinations of pulse amplitude and duration, followed by imaging using pulses with lower pulse amplitude and duration. In this case, carbon nano-tubes that have become detached from their marker by the test pulse will subsequently leave an area of body 19 where they were bound to receptors, or where they would have become bound. By comparing the images obtained after test pulses with different energy, or combination of amplitude and duration, a relative difference decrease in the concentration of the carbon nanotubes can be used to determine a threshold energy at which no more than a predetermined fraction of the carbon nanotubes becomes detached. In an embodiment, imaging is performed with pulses that have an energy below this threshold. This makes it possible to perform a time sequence of measurements that is not significantly affected by detachment. But of course a higher energy may be used, for example if only one image is needed for a set of carbon nano-tubes, or a plurality of images in close temporal proximity.

In an embodiment a pulse at the wavelength of a set of carbon nano-tubes with higher energy than the energy of the pulses used for imaging using that set may be transmitted to body 19 to detach markers from carbon nano tubes on purpose. For example, such a higher energy pulse may be transmitted before obtaining one or more images using lower energy pulses at that wavelength. The higher energy pulse may have at least twice and preferably at least ten times the energy of the pulses used for imaging. This may be used as a “reset”, to promote removal of the carbon nano tubes, before obtaining an image using later supplied carbon nano-tubes of the set. In another embodiment, it may be used to determine a rate of release by measuring temporal changes in images obtained with pulses at this wavelength. In an embodiment the higher energy pulse may comprise radiation at the wavelengths associated with a plurality of the sets, having said higher energy for each of the wavelengths individually.

In a further embodiment, a plurality of further sets of carbon nano-tubes that contain or are attached to drugs may be supplied to body 19. Such carbon nanotubes are known per se from US 2008193490. In the present case, a plurality of different drugs is used, each supplied by a different further set of carbon nanotubes. Examples of possible substances that could be added as drugs are small molecule drugs including antitumor drugs, Plasmid DNA, Short interfering RNA (siRNA). Nucleotide sequences and Peptide sequences. Each such further set has a respective associated wavelength and associated drug. The carbon nano-tubes in the further set have an absorption peak at the associated wavelength of the further set. Different further sets have different associated wavelengths and different associated drugs.

In operation, the release of a selected drug is mediated by transmitting a high energy pulse substantially at the wavelength that is associated with the further set. The minimum required high energy may be determined experimentally. This makes it possible to apply a plurality of drugs to a patient and to select one or more of these drugs for release after supplying the plurality of drugs. When the further sets of carbon nano-tubes are present in body 19 at the same time, for example after supplying them at the same time, the images obtained with sets of carbon-nanotubes with different markers may be used to select which one or more of the drugs should be released by using the high energy pulse. Furthermore, the images may be used to select areas in the body where the energy of the high energy pulse should be concentrated, other areas receiving less energy. Thus, a position dependent release is possible.

In a further embodiment the sets and the further sets may coincide, that is a set of carbon nano-tubes that is associated with a wavelength may contain both carbon nano-tubes that carry a marker and carbon nano-tubes than supply a drug. The same carbon nano-tubes may both carry markers and drugs, or different carbon nano-tubes associated with the same wavelength may carry markers and drugs respectively. In this way a set can be used both for selective imaging and selective release.

The further sets can also be applied without application of the sets used for imaging and without imaging. Thus method is provided wherein further sets of carbon nanotubes are used, respective ones of the further sets each comprising carbon nanotubes carrying respective different releasable substance, the carbon nanotubes of the respective one of the further sets having a respective geometry giving rise to an absorption peak for electromagnetic radiation at a wavelength different from the wavelengths of the absorption peaks corresponding to the chiral number of the carbon nanotubes in the other ones of the further sets, the method comprising
administering a combination of carbon nanotubes from each of said sets to the body, in combination with a combination of carbon nanotubes from each of said further sets;
selectively releasing said substances from the carbon nanotubes from a selected one or ones of the further sets by transmitting electromagnetic radiation to the body, substantially at the wavelengths of the absorption peaks of the selected one or ones of the further sets.

The mix of further sets may be realized first mixing a selection of separate solutions that each contains one of the releasable substances carried by carbon nanotubes that absorb electromagnetic radiation in an absorption peak distinct from absorption peaks of carbon nanotubes carrying markers in the other solutions. The result of this mixing may be fed to body 19. Alternatively, the mix may be applied by applying the solutions separately to body 19 with a relative timing such that the carbon nanotubes from different solutions will be present simultaneously in body 19. The set of solutions forms a kit of parts from which a mix can be composed.

Although embodiments have been described wherein pulses of electromagnetic radiation in the infrared range (wavelengths of 0.7-1.1 micrometer) are used, it should be appreciated that other wavelengths may be used. For example wavelengths in a microwave range, deep infrared, optical wavelengths etc. Use of radiation with wavelengths of 0.7-1.1 micrometer is advantageous because this radiation easily penetrates the human body. Instead of pulses modulated electromagnetic radiation may be used, for example with a periodically modulated amplitude with a period that is significantly longer than that of the ultrasound, or other modulation patterns. When modulated electromagnetic radiation is used, the use of mutually different modulation for radiation at different wavelengths may be used to distinguish different sets. Thus, instead of time division multiplexing of pulses of different wavelength, modulation frequency division multiplexing or code division multiplexing (CDMA) may be used.

Although embodiments have been described wherein each set of carbon nanotubes (i.e. all carbon nanotubes that absorb at the same wavelength) carry the same marker that is specific for the set, it should be appreciated that a set may contain carbon nanotubes that carry mutually different markers. When these markers are different from those in other sets, useful different images will be obtained. In fact, it may suffice that the combination of concentrations of carbon nanotubes in the set that carry mutually different markers is different from the combination of concentrations of carbon nanotubes in other sets. In this case the images can still be used to provide different information.

For example, the carbon nanotubes of a first set may contain a concentration Ca (=fraction of all carbon nanotubes in the set) of carbon nanotubes that carry a maker A and a concentration Cb of carbon nanotubes that carry a maker B. In this example, the carbon nanotubes of a second set may contain a concentration Ca′ of carbon nanotubes that carry a maker A and a concentration Cb′ of carbon nanotubes that carry a maker B, with Ca unequal to Ca and Cb unequal to Cb′. In this case irradiation with electromagnetic radiation at the wavelengths of the first and second set may result in different images, or at least images that depend in different ways on receptor densities, due to the concentration differences. These images provide independent information about the density of receptors for the markers A and B. Use of sets that each contains substantially only carbon nanotubes with a set specific type of marker is just an extreme case of use of different combinations of concentrations. Use of a set specific type of marker may provide for more accurate information about density of receptors. Although embodiments have been described wherein the body 19 of material is the body of a human or animal, to which the mix of set of carbon nano-tubes has been supplied, it should be appreciated that any type of body could be used. For example, a plant may be used as body, a volume of food or micro-organisms, a biopt, a machine or other industrial structure wherein different receptor materials may be present for example as pollution etc.

Although embodiments have been described wherein detection of the effect of the electromagnetic radiation is performed by means of ultrasound detection, it should be appreciated that other detection techniques may be used. Inelastic scattering from the carbon nanotubes ((Raman) may be detected for example.

Processing system 14 may be implemented as a single programmable computer, or by means of a plurality of programmable computers. Part or all of processing system 14 may be realized by means of circuits that are specifically designed to perform the described functions.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An imaging method that uses a plurality of sets of carbon nanotubes, respective ones of the sets each comprising carbon nanotubes carrying markers for selectively binding to a respective receptor in a body of material, different from markers of the carbon nanotubes in other ones of the sets, or in a different combination of concentrations of markers as in the other ones of the sets, the carbon nanotubes of the respective one of the sets having a respective geometry giving rise to an absorption peak for electromagnetic radiation at a wavelength different from the wavelengths of the absorption peaks corresponding to the geometry of the carbon nanotubes in the other ones of the sets, the method comprising
transmitting electromagnetic radiation to the body, substantially at the wavelengths of the absorption peaks of the sets, multiplexed with each other;
detecting a response to absorption of the transmitted electromagnetic radiation;
detecting amplitudes of ultrasound in said response to absorption of the transmitted electromagnetic radiation at respective ones of said frequencies to form images of the absorption as a function of position for respective ones of the plurality of the wavelengths;
forming images of the absorption as a function of position in the body for respective ones of the plurality of the wavelengths, and/or an image dependent on the absorption as a function of position in the body for a selected combination of the plurality of the wavelengths and where in the carbon nanotbues that carry the markers of different ones of the sets have mutually different chiral numbers.

2. (canceled)

3. An imaging method according to claim 1, comprising administering a combination of carbon nanotubes from each of said sets to the body.

4. An imaging method according to claim 1, wherein said detected response comprises an amplitude of ultrasound waves excited by the absorption of the transmitted electromagnetic radiation.

5. An imaging method according to claim 1, wherein the electromagnetic radiation is transmitted in pulses, the wavelengths being multiplexed by transmitting pulses with electromagnetic radiation substantially at the wavelengths of the carbon nanotubes of respective ones of the sets at mutually different time points.

6. An imaging method according to claim 5, comprising transmitting a further pulse at the wavelength of at least one of the sets, with a higher energy than said pulses from which said detection of the response to absorption is performed at least at said wavelength of the at least one of the sets.

7. An imaging method according to claim 1, comprising the use of further sets of carbon nanotubes, respective ones of the further sets each comprising carbon nanotubes carrying respective different releasable substance, the carbon nanotubes of the respective one of the further sets having a respective geometry giving rise to an absorption peak for electromagnetic radiation at a wavelength different from the wavelengths of the absorption peaks corresponding to the chiral number of the carbon nanotubes in the other ones of the further sets, the method comprising
administering a combination of carbon nanotubes from each of said sets to the body, in combination with a combination of carbon nanotubes from each of said further sets;
selectively releasing said substances from the carbon nanotubes from a selected one or ones of the further sets by transmitting electromagnetic radiation to the body, substantially at the wavelengths of the absorption peaks of the selected one or ones of the further sets and wherein the carbon nanotubes that carry the -leasable substances of different ones of the further sets have mutually different chiral numbers.

8. (canceled)

9. An imaging method according to claim 7, wherein the releasable substances are selected from the group consisting of small molecule drugs, including antitumor drugs, Plasmid DNA, Short interfering RNA (siRNA), Nucleotide sequences and Peptide sequences

10. An imaging method according to claim 7, wherein the wavelengths of the transmitted electromagnetic radiation lie between 0.7 and 1.1 micrometer.

11. An imaging method according to claim 7, wherein the markers include markers selected from the group consisting of monoclonal antibodies, peptides, vitamins, aptamers.

12. An imaging system, comprising
an electromagnetic radiation source;
an array of ultrasound detectors;
a combination of carbon nanotubes from a plurality of sets of carbon nanotubes, respective ones of the sets each comprising carbon nanotubes carrying markers for selectively binding to a respective receptor in a body of material, different from markers of the carbon nanotubes in other ones of the sets, or in a different combination of concentrations of markers as in the other ones of the sets, the carbon nanotubes of the respective one of the sets having a respective geometry giving rise to an absorption peak for electromagnetic radiation at a wavelength different from the wavelengths of the absorption peaks corresponding to the geometry of the carbon nanotubes in the other ones of the sets,
a processing system configured to cause the electromagnetic radiation source to transmit electromagnetic radiation, substantially at the wavelengths of the absorption peaks of the sets, multiplexed with each other; to receive detection signals from the ultrasound detectors; and to use amplitudes of detected ultrasound in response to absorption of the transmitted electromagnetic radiation at respective ones of said frequencies to form images of the absorption as a function of position for respective ones of the plurality of the wavelengths, and/or an image dependent on the absorption as a function of position in the body for a selected combination of the plurality of the wavelengths and wherein the carbon nanotubes that carry the markers of different ones of the sets have mutually different chiral numbers.

13. (canceled)

14. A composition comprising a combination of carbon nanotubes from a plurality of sets of carbon nanotubes, respective ones of the sets each comprising carbon nanotubes carrying markers for selectively binding to a respective receptor in a body of material, different from markers of the carbon nanotubes in the other ones of the sets, or in a different combination of concentration of markers as in the other ones of the sets, the carbon nanotubes of the respective one of the sets having a respective geometries giving rise to
(i) an absorption peak for electromagnetic radiation at a wavelength different from the wavelengths of the absorption peaks corresponding to the chiral number of the carbon nanotubes in the other ones of the sets and
(ii) ultrasound emission in response to said absorption of the transmitted electromagnetic radiation at respective ones of said frequencies to form images of the absorption as a function of position for respective ones of the plurality of the wavelengths.

15. A kit of parts, comprising a plurality of solutions each solution comprising carbon nanotubes carrying markers for selectively binding to a respective different receptor, or in a respective different combination of concentrations of markers, the carbon nanotubes of the respective one of the sets having a respective different geometries giving rise to
(i) an absorption peak for electromagnetic radiation at a wavelength different from the wavelengths of the absorption peaks corresponding to the chiral number of the carbon nanotubes in the other ones of the sets and
(ii) ultrasound emission in response to said absorption of the transmitted electromagnetic radiation at respective ones of said frequencies to form images of the absorption as a function of position for respective ones of the plurality the wavelengths.
